(19) United States
(12) Patent Application Publication
(10) Pub. No.: US 2006/0188470 A1
(43) Pub. Date: Aug. 24, 2006

(54) PES 2000 plus folic acid

(76) Inventor: William H. Livingston, Artesia, NM (US)

Correspondence Address: WILLIAM HUBERT LIVINGSTON, R223 NORTH 13TH STREET, ARTESIA, NM, 88210, US

(21) Appl. No.: 11/062,932

(22) Filed: Feb. 22, 2005

(51) Int. Cl. A61K 31/765; A61K 31/525

(52) U.S. Cl. 424/78.38; 514/251

(57) ABSTRACT

1. A therapeutic dose of PES 2000 when taken orally by animals or humans helps to control and prevent epilepsy.

### CROSS-REFERENCE OR DRAWINGS

none.

### SUMMARY

New and useful discovery that “PES 2000 plus Folic Acid” containing per capsule, Folic Acid 10 mg, Succinic Acid 200 mg, Glutamic Acid 50 mg, Calcium Succinate 50 mg.

This combination has the ability to help prevent and control Epilepsy, when taken orally, on a daily basis by humans and animals.

### BACKGROUND OF INVENTION

Method of discovery is as follows:

“Primary Energy Source,(PES 2000), Plus Folic Acid”, containing a therapeutic dose, when taken orally controlled Epilepsy in both humans and animals.

### DESCRIPTION AND ADVANTAGE OF THE INVENTION

Epilepsy was prevented and controlled in both human and animals

I claim:

1. A therapeutic dose of “PES 2000 plus Folic Acid” helps to prevent and control Epilepsy in both humans and animals.
